(19) United States
(12) Patent Application Publication
(10) Pub. No.: US 2024/0226172 A9
(43) Pub. Date: Jul. 11, 2024

(54) MESENCHYMAL STEM CELL THERAPY OF EPILEPSY AND SEIZURE DISORDERS

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US)

(73) Assignee: Therapeutic Solutions International, Inc., Oceanside, CA (US)

(21) Appl. No.: 18/493,694

(22) Filed: Oct. 24, 2023

Related U.S. Application Data
(60) Provisional application No. 63/418,861, filed on Oct. 24, 2022.

(51) Int. Cl. A61K 35/28; A61P 25/08; C12N 5/0775

(52) U.S. Cl. CPC A61K 35/28; A61P 25/08; C12N 5/0662

(57) ABSTRACT

Disclosed are novel compositions of matter and treatment methods for reducing and/or reversing epilepsy through administration of mesenchymal stem cells in order to induce immune modulation and/or regenerative processes. In one embodiment umbilical cord mesenchymal stem cells are administered to a patient suffering from epilepsy at a concentration and frequency sufficient to inhibit neuronal hyperactivation and/or reduce neuroinflammatory status of the patient

### CROSS-REFERENCE TO RELATED APPLICATIONS

This application claims priority to U.S. Provisional Application No. 63/418,861, titled “Mesenchymal Stem Cell Therapy of Epilepsy and Seizure Disorders”, filed Oct. 24, 2022, which is hereby incorporated by reference in its entirety.

### FIELD OF THE INVENTION

The teachings herein relate to the use of regenerative cells for the treatment and prevention of epilepsy and other seizure disorders.

### BACKGROUND OF THE INVENTION

Temporal lobe epilepsy (TLE) is a chronic disease characterized by spontaneous, progressive seizures. In many patients, TLE is initiated by brain injury, strokes, tumors or status epilepticus (SE), which is often followed by a latency period of 5-10 years before the onset of spontaneous recurrent motor seizure (SRMS). SE is defined as a continuous seizure activity lasting more than 5 min. SRMS is the repeated unprovoked seizures that occurs in the chronic phase of epilepsy. The main physiopathological findings observed in TLE are neuronal loss, reactive gliosis, aberrant mossy fiber sprouting and spontaneous recurrent motor seizure (SRMS). Antiepileptic drugs fail to well-control seizures in approximately 30% of epilepsy patients.

There is a significant need in the field to develop means of controlling seizures and/or reversing epilepsy.

### SUMMARY

Preferred embodiments include methods of inhibiting or reversing epilepsy or seizure syndrome by administration of a regenerative cell at a sufficient frequency and concentration to induce a therapeutic effect in a patient in need.

Preferred methods include embodiments wherein administration of an anti-inflammatory and/or anti-epileptic drug is also administered.

Preferred methods include embodiments wherein said regenerative cells are mesenchymal stem cell are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; 1) cord blood; m) omentum; n) muscle; o) amniotic membrane; o) periventricular fluid; and p) placental tissue.

Preferred methods include embodiments wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein said mesenchymal stem cells do not express substantial levels of HLA-DR, CD117, and CD45.

Preferred methods include embodiments wherein said mesenchymal stem cells are generated from a pluripotent stem cell.

Preferred methods include embodiments wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

Preferred methods include embodiments wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred methods include embodiments wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

Preferred methods include embodiments wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred methods include embodiments wherein said somatic cell nuclear transfer derived stem cells possess a phenotype negative for SSEA-1 and positive for SSEA-3, SSEA-4, TRA-1-60, TRA-1-81, and alkaline phosphatase.

Preferred methods include embodiments wherein said mesenchymal stem cells are differentiated from a pluripotent stem cell source through culture in the presence of an inhibitor of the SMAD-2/3 pathway.

Preferred methods include embodiments wherein said mesenchymal stem cells are differentiated from a pluripotent stem cell source through culture in the presence of an inhibitor nucleic acid targeting the SMAD-2/3 pathway.

Preferred methods include embodiments wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

Preferred methods include embodiments wherein said inhibitor of the SMAD-2/3 pathway is a small molecule inhibitor.

Preferred methods include embodiments wherein said small molecule inhibitor is SB-431542.

Preferred methods include embodiments wherein a selection process is used to enrich for mesenchymal stem cells differentiated from said pluripotent stem cell population.

Preferred methods include embodiments wherein said enrichment method comprises of positively selecting for cells expressing a marker associated with mesenchymal stem cells.

Preferred methods include embodiments wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein the subject is known to have Asherman's syndrome.

Preferred methods include embodiments wherein the subject has endometrial atrophy that is resistant to hormonal or other treatments.

Preferred methods include embodiments wherein the subject has had one or more prior embryo implantation failures.

Preferred methods include embodiments wherein the regenerative cells are prepared by administering to the subject an agent to mobilize regenerative cells from bone marrow into peripheral blood of the subject; and isolating said regenerative cells from peripheral blood of the subject.

Preferred methods include embodiments wherein the agent to mobilize regenerative cells is granulocyte colony-stimulating factor (G-CSF).

Preferred methods include embodiments wherein the agent to mobilize regenerative cells is granulocyte monocyte colony-stimulating factor (GM-CSF).

Preferred methods include embodiments wherein the agent to mobilize regenerative cells is Leukemia Inhibiting Factor (LIF).

Preferred methods include embodiments wherein the agent to mobilize regenerative cells is HGF-1.

Preferred methods include embodiments wherein the agent to mobilize regenerative cells is FGF-1.

Preferred methods include embodiments wherein the agent to mobilize regenerative cells is FGF-2.

Preferred methods include embodiments wherein the agent to mobilize regenerative cells is AMD-3100.

Preferred methods include embodiments wherein the agent to mobilize regenerative cells is M=CSF.

Preferred methods include embodiments wherein the agent to mobilize regenerative cells is ozone therapy.

Preferred methods include embodiments wherein the agent to mobilize regenerative cells is IL-2.

Preferred methods include embodiments wherein the agent to mobilize regenerative cells is FLT-3 ligand.

Preferred methods include embodiments wherein the agent to mobilize regenerative cells is TNF alpha.

Preferred methods include embodiments wherein the agent to mobilize regenerative cells is hCG.

Preferred methods include embodiments wherein the agent to mobilize regenerative cells is hyperbaric oxygen.

Preferred methods include embodiments wherein the agent to mobilize regenerative cells is BDNF.

Preferred methods include embodiments wherein the agent to mobilize regenerative cells is NGF-1.

Preferred methods include embodiments wherein the agent to mobilize regenerative cells is VEGF.

Preferred methods include embodiments wherein the regenerative cells are isolated from peripheral circulation of the subject by apheresis using an antibody that has selective affinity to said regenerative cells.

Preferred methods include embodiments wherein said regenerative cells are isolated with an antibody capable of binding cells expressing CD31.

Preferred methods include embodiments wherein said regenerative cells are isolated with an antibody capable of binding cells expressing CD33.

Preferred methods include embodiments wherein said regenerative cells are isolated with an antibody capable of binding cells expressing CD34.

Preferred methods include embodiments wherein said regenerative cells are isolated with an antibody capable of binding cells expressing CD133.

Preferred methods include embodiments wherein said regenerative cells are isolated with an antibody capable of binding cells expressing CD34.

Preferred methods include embodiments wherein said regenerative cells are isolated with an antibody capable of binding cells expressing CD31 and CD34.

Preferred methods include embodiments wherein said regenerative cells are isolated with an antibody capable of binding cells expressing CD31 and CD33.

Preferred methods include embodiments wherein said regenerative cells are isolated with an antibody capable of binding cells expressing CD31 and VEGF receptor.

Preferred methods include embodiments wherein said regenerative cells are isolated with an antibody capable of binding cells expressing CD31 and HGF-1 receptor.

Preferred methods include embodiments wherein said regenerative cells are isolated with an antibody capable of binding cells expressing CD31 and stem cell factor.

Preferred methods include embodiments wherein said regenerative cells are isolated with an antibody capable of binding cells expressing TLR-4 and CD34.

Preferred methods include embodiments wherein said regenerative cells are isolated with an antibody capable of binding cells expressing TLR-4 and CD33.

Preferred methods include embodiments wherein said regenerative cells are isolated with an antibody capable of binding cells expressing TLR-4 and VEGF receptor.

Preferred methods include embodiments wherein said regenerative cells are isolated with an antibody capable of binding cells expressing TLR-4 and HGF-1 receptor.

Preferred methods include embodiments wherein said regenerative cells are isolated with an antibody capable of binding cells expressing TLR-4 and stem cell factor.

Preferred methods include embodiments wherein said regenerative cells are isolated with an antibody capable of binding cells expressing TLR-4 and CD90.

Preferred methods include embodiments wherein said regenerative cells are isolated with an antibody capable of binding cells expressing TLR-4 and CD13.

Preferred methods include embodiments wherein said regenerative cells are isolated with an antibody capable of binding cells expressing TLR-4 and CD29.

Preferred methods include embodiments wherein said regenerative cells are isolated with an antibody capable of binding cells expressing TLR-4 and CD44.

Preferred methods include embodiments wherein said regenerative cells are isolated with an antibody capable of binding cells expressing TLR-4 and CD71.

Preferred methods include embodiments wherein said regenerative cells are isolated with an antibody capable of binding cells expressing TLR-4 and CD73.

Preferred methods include embodiments wherein said regenerative cells are isolated with an antibody capable of binding cells expressing TLR-4 and CD105.

Preferred methods include embodiments wherein said regenerative cells are isolated with an antibody capable of binding cells expressing TLR-4 and CD166.

Preferred methods include embodiments wherein said regenerative cells are isolated with an antibody capable of binding cells expressing TLR-4 and STRO-1.

Preferred methods include embodiments wherein said regenerative cells are isolated with an antibody capable of binding cells expressing TLR-4 and STRO-4.

Preferred methods include embodiments wherein said regenerative cells are isolated with an antibody capable of binding cells expressing TLR-4 and TNF receptor p55.

Preferred methods include embodiments wherein said regenerative cells are isolated with an antibody capable of binding cells expressing TLR-4 and TNF receptor p75.

Preferred methods include embodiments wherein said regenerative cells are isolated with an antibody capable of binding cells expressing TLR-4 and CD227.

Preferred methods include embodiments wherein said regenerative cells are isolated with an antibody capable of binding cells expressing c-kit and CD34.

Preferred methods include embodiments wherein said regenerative cells are isolated with an antibody capable of binding cells expressing c-kit and CD33.

Preferred methods include embodiments wherein said regenerative cells are isolated with an antibody capable of binding cells expressing c-kit and VEGF receptor.

Preferred methods include embodiments wherein said regenerative cells are isolated with an antibody capable of binding cells expressing c-kit and HGF-1 receptor.

Preferred methods include embodiments wherein said regenerative cells are isolated with an antibody capable of binding cells expressing c-kit and stem cell factor.

Preferred methods include embodiments wherein said regenerative cells are isolated with an antibody capable of binding cells expressing c-kit and CD90.

Preferred methods include embodiments wherein said regenerative cells are isolated with an antibody capable of binding cells expressing c-kit and CD13.

Preferred methods include embodiments wherein said regenerative cells are isolated with an antibody capable of binding cells expressing c-kit and CD29.

Preferred methods include embodiments wherein said regenerative cells are isolated with an antibody capable of binding cells expressing c-kit and CD44.

Preferred methods include embodiments wherein said regenerative cells are isolated with an antibody capable of binding cells expressing c-kit and CD71.

Preferred methods include embodiments wherein said regenerative cells are isolated with an antibody capable of binding cells expressing c-kit and CD73.

Preferred methods include embodiments wherein said regenerative cells are isolated with an antibody capable of binding cells expressing c-kit and CD105.

Preferred methods include embodiments wherein said regenerative cells are isolated with an antibody capable of binding cells expressing c-kit and CD166.

Preferred methods include embodiments wherein said regenerative cells are isolated with an antibody capable of binding cells expressing c-kit and STRO-1.

Preferred methods include embodiments wherein said regenerative cells are isolated with an antibody capable of binding cells expressing c-kit and STRO-4.

Preferred methods include embodiments wherein said regenerative cells are isolated with an antibody capable of binding cells expressing c-kit and TNF receptor p55.

Preferred methods include embodiments wherein said regenerative cells are isolated with an antibody capable of binding cells expressing c-kit and TNF receptor p75.

Preferred methods include embodiments wherein said regenerative cells are isolated with an antibody capable of binding cells expressing c-kit and CD227.

Preferred methods include embodiments wherein said regenerative cells are isolated with an antibody capable of binding cells expressing OCT-4 and CD34.

Preferred methods include embodiments wherein said regenerative cells are isolated with an antibody capable of binding cells expressing OCT-4 and CD33.

Preferred methods include embodiments wherein said regenerative cells are isolated with an antibody capable of binding cells expressing OCT-4 and VEGF receptor.

Preferred methods include embodiments wherein said regenerative cells are isolated with an antibody capable of binding cells expressing OCT-4 and HGF-1 receptor.

Preferred methods include embodiments wherein said regenerative cells are isolated with an antibody capable of binding cells expressing OCT-4 and stem cell factor.

Preferred methods include embodiments wherein said regenerative cells are isolated with an antibody capable of binding cells expressing OCT-4 and CD90.

Preferred methods include embodiments wherein said regenerative cells are isolated with an antibody capable of binding cells expressing OCT-4 and CD13.

Preferred methods include embodiments wherein said regenerative cells are isolated with an antibody capable of binding cells expressing OCT-4 and CD29.

Preferred methods include embodiments wherein said regenerative cells are isolated with an antibody capable of binding cells expressing OCT-4 and CD44.

Preferred methods include embodiments wherein said regenerative cells are isolated with an antibody capable of binding cells expressing OCT-4 and CD71.

Preferred methods include embodiments wherein said regenerative cells are isolated with an antibody capable of binding cells expressing OCT-4 and CD73.

A method of preventing or treating endometrial atrophy comprising of administering regenerative cells combined with endometrial stimulating factors.

Preferred methods include embodiments wherein said regenerative cells are mesenchymal stem cells.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45;

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said umbilical cord tissue-derived cells are capable of differentiating into one or more lineages selected from a group comprising of; a) ectoderm; b) mesoderm, and; c) endoderm.

Preferred methods include embodiments wherein said bone marrow derived mesenchymal stem cells possess markers selected from a group comprising of: a) CD73; b) CD90; and c) CD105.

Preferred methods include embodiments wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

Preferred methods include embodiments wherein said bone marrow derived mesenchymal stem cell is a mesenchymal stem cell progenitor cell.

Preferred methods include embodiments wherein said mesenchymal progenitor cells are a population of bone marrow mesenchymal stem cells enriched for cells containing STRO-1

Preferred methods include embodiments wherein said mesenchymal progenitor cells express both STRO-1 and VCAM-1.

Preferred methods include embodiments wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Preferred methods include embodiments wherein said bone marrow mesenchymal stem cells lack expression of CD14, CD34, and CD45.

Preferred methods include embodiments wherein said STRO-1 expressing cells are positive for a marker selected from a group comprising of: a) VACM-1; b) TKY-1; c) CD146 and; d) STRO-2

Preferred methods include embodiments wherein said bone marrow mesenchymal stem cell express markers selected from a group comprising of: a) CD13; b) CD34; c) CD56 and; d) CD117

Preferred methods include embodiments wherein said bone marrow mesenchymal stem cells do not express CD10.

Preferred methods include embodiments wherein said bone marrow mesenchymal stem cells do not express CD2, CD5, CD14, CD19, CD33, CD45, and DRII.

Preferred methods include embodiments wherein said bone marrow mesenchymal stem cells express CD13,CD34, CD56, CD90, CD117 and nestin, and which do not express CD2, CD3, CD10, CD14, CD16, CD31, CD33, CD45 and CD64.

Preferred methods include embodiments wherein said skeletal muscle stem cells express markers selected from a group comprising of: a) CD13; b) CD34; c) CD56 and; d) CD117

Preferred methods include embodiments wherein said skeletal muscle mesenchymal stem cells do not express CD10.

Preferred methods include embodiments wherein said skeletal muscle mesenchymal stem cells do not express CD2, CD5, CD14, CD19, CD33, CD45, and DRII.

Preferred methods include embodiments wherein said bone marrow mesenchymal stem cells express CD13,CD34, CD56, CD90, CD117 and nestin, and which do not express CD2, CD3, CD10, CD14, CD16, CD31, CD33, CD45 and CD64.

Preferred methods include embodiments wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Preferred methods include embodiments wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Preferred methods include embodiments wherein said subepithelial umbilical cord derived mesenchymal stem cells express CD29, CD73, CD90, CD166, SSEA4, CD9, CD44, CD146, and CD105.

Preferred methods include embodiments wherein said subepithelial umbilical cord derived mesenchymal stem cells do not express CD45, CD34, CD14, CD79, CD106, CD86, CD80, CD19, CD117, Stro-1, and HLA-DR.

Preferred methods include embodiments wherein said subepithelial umbilical cord derived mesenchymal stem cells are positive for SOX2.

Preferred methods include embodiments wherein said subepithelial umbilical cord derived mesenchymal stem cells are positive for OCT4.

Preferred methods include embodiments wherein said subepithelial umbilical cord derived mesenchymal stem cells are positive for OCT4 and SOX2.

### BRIEF DESCRIPTION OF THE DRAWINGS

FIG. 1 is a bar graph showing the therapeutic effects of (1) Pilocarpine, (2) Pilocarpine+UC MSC, and (3) Pilocarpine+Jadi Cell cells on seizures in mice.

### DETAILED DESCRIPTION OF THE INVENTION

The invention teaches the use of mesenchymal stem cells, particularly but not exclusively cells known as “Jadi Cell™”. JadiCell™ is a trademark for a population of plastic adherent, umbilical cord derived, mesenchymal stem cells positive for the following cellular markers: CD7, CD11, CD90, CD105, CD133, interleukin 1 receptor, interleukin 3 receptor, interleukin 6 receptor, interleukin 13 receptor, interleukin 17 receptor, interleukin 17F receptor, and interleukin 10 receptor.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium. In some embodiments the invention teaches the use of conditioned media, or concentrated conditioned media, or exosomes isolated from conditioned media of EPC or MSC to promote tolerogenesis.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLl, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78). In some embodiments of the invention mesenchymal stem cells are selected for placental expression of OCT-4. In other embodiments, OCT-4 expression is used as a means of identifying cells for culture and expansion subsequent to exposure to various culture conditions.

In a presently preferred embodiment, the isolation procedure also utilizes an enzymatic digestion process. Enzymes are used to dissociated tissue to extract cellular populations that are subsequently harvested and grown for isolation of fetal derived mesenchymal stem cells. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activities is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells. Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer. Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD45, CD117, CD141, or HLA-DR,DP, DQ.

The dose of placental MSC appropriate to be used in accordance with various embodiments of the invention will depend on numerous factors. It may vary considerably for different circumstances. The parameters that will determine optimal doses of placental MSC to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the Placental MSC are syngeneic, autologous, allogeneic, or xenogeneic; their potency (specific activity); the site and/or distribution that must be targeted for the Placental MSC to be effective; and such characteristics of the site such as accessibility to Placental MSC and/or engraftment of Placental MSC. Additional parameters include co-administration with Placental MSC of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of Placental MSC outweighs the advantages of the increased dose. The optimal dose of placental MSC for some embodiments will be in the range of doses used for autologous, mononuclear bone marrow transplantation. For fairly pure preparations of placental MSC, optimal doses in various embodiments will range from 10.sup.4 to 10.sup.8 placental MSC cells/kg of recipient mass per administration. In some embodiments the optimal dose per administration will be between 10.sup.5 to 10.sup.7 placental MSC cells/kg. In many embodiments the optimal dose per administration will be 5.times.10.sup.5 to 5.times.10.sup.6 placental MSC cells/kg. By way of reference, higher doses in the foregoing are analogous to the doses of nucleated cells used in autologous mononuclear bone marrow transplantation. Some of the lower doses are analogous to the number of CD34.sup.+ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations. In various embodiments, Placental MSC may be administered in an initial dose, and thereafter maintained by further administration of Placental MSC. Placental MSC may be administered by one method initially, and thereafter administered by the same method or one or more different methods. The subject's PLACENTAL MSC levels can be maintained by the ongoing administration of the cells. Various embodiments administer the Placental MSC either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer Placental MSC. In some embodiments Placental MSC are administered to a subject in one dose. In others Placental MSC are administered to a subject in a series of two or more doses in succession. In some other embodiments wherein Placental MSC are administered in a single dose, in two doses, and/or more than two doses, the doses may be the same or different, and they are administered with equal or with unequal intervals between them. Placental MSC may be administered in many frequencies over a wide range of times. In some embodiments, placental MSC are administered over a period of less than one day. In other embodiment they are administered over two, three, four, five, or six days. In some embodiments Placental MSC are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

### EXAMPLES

### Example 1: Treatment of Mouse Model of Epilepsy

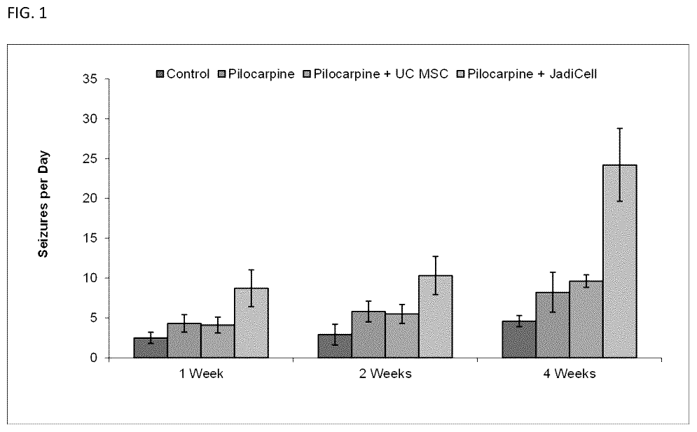

C57 BL/6 male mice treated with anti-muscarinic drug scopolamine (1 mg/kg, intraperitoneal; S2250; Sigma) to minimize peripheral muscarinic effects. Thirty minutes after scopolamine, mice were injected with pilocarpine (330 mg/kg, intraperitoneally; P6503; Sigma) to induce SE. Mice developed SE within 5-30 min. To limit the duration of SE and the extent of the damage in the hippocampus, diazepam (10-20 mg/kg; CCPC) was injected intraperitoneally 90 min after the onset of SE. Mice injected with 500,000 control UC-MSC or 500,000 Jadi Cell™. Seizures quantified by filming. Results are shown in FIG. 1

### Example 2: Case Report

The 65 year old patient had a history of epilepsy since the age of 28, leading to a life of crippling seizures and severe chronic tinnitus. The patient preferred to live in darkness for years due to any form of sound triggering violent seizures. The patient reported that he had not seen a movie, read a book or stepped out of his home for 35 years. We treated this patient with four regimens of Jadi Cell™ stem cells at 50 million cells per injection. In the first follow-up on Day 3 after treatment, he was feeling better and had not suffered a single seizure. By Day 7, he was watching his first movie and had begun to read his first book in decades. As of Day 20 after treatment, he was actually building a concrete flower container in his backyard.

1. A method of inhibiting or reversing epilepsy or seizure syndrome in a patient in need by administration of a regenerative cell at a sufficient frequency and concentration to induce a therapeutic effect.

2. The method of claim 2, wherein administration of an anti-inflammatory and/or anti-epileptic drug is also administered.

3. The method of claim 2, wherein said regenerative cells are mesenchymal stem cell are derived from tissue selected from the group consisting of: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

4. The method of claim 3, wherein said mesenchymal stem cells express a marker selected from the group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

5. The method of claim 4, wherein said mesenchymal stem cells do not express substantial levels of HLA-DR, CD117, and CD45.

6. The method of claim 3, wherein said mesenchymal stem cells are generated from a pluripotent stem cell.

7. The method of claim 6, wherein said pluripotent stem cell is selected from the group consisting of: a) an embryonic stem cell; b) an inducible pluripotent stem cell; c) a parthenogenic stem cell; and d) a somatic cell nuclear transfer derived stem cell.

8. The method of claim 7, wherein said embryonic stem cell population expresses genes selected from the group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

9. The method of claim 7, wherein said inducible pluripotent stem cell possesses markers selected from the group consisting of: CD10, CD13, CD44, CD73, CD90, PDGFr-alpha, PD-L2, and HLA-A,B,C and possesses ability to undergo at least 40 doublings in culture, while maintaining a normal karyotype upon passaging.

10. The method of claim 7, wherein said parthenogenic stem cells wherein said parthenogenically derived stem cells are generated by addition of a calcium flux inducing agent to activate an oocyte followed by enrichment of cells expressing markers selected from the group consisting of: SSEA-4, TRA 1-60 and TRA 1-81.

11. The method of claim 7, wherein said somatic cell nuclear transfer derived stem cells possess a phenotype negative for SSEA-1 and positive for SSEA-3, SSEA-4, TRA-1-60, TRA-1-81, and alkaline phosphatase.

12. The method of claim 6, wherein said mesenchymal stem cells are differentiated from a pluripotent stem cell source through culture in the presence of an inhibitor of the SMAD-2/3 pathway.

13. The method of claim 12, wherein said mesenchymal stem cells are differentiated from a pluripotent stem cell source through culture in the presence of an inhibitor nucleic acid targeting the SMAD-2/3 pathway.

14. The method of claim 13, wherein said nucleic acid inhibitor is selected from the group consisting of: a) an antisense oligonucleotide; b) a hairpin loop short interfering RNA; c) a chemically synthesized short interfering RNA molecule; and d) a hammerhead ribozyme.

15. The method of claim 13, wherein said inhibitor of the SMAD-2/3 pathway is a small molecule inhibitor.

16. The method of claim 15, wherein said small molecule inhibitor is SB-431542.

17. The method of claim 6, wherein a selection process is used to enrich for mesenchymal stem cells differentiated from said pluripotent stem cell population.

18. The method of claim 17, wherein said marker of mesenchymal stem cells is selected from the group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

19. The method of claim 17, wherein said mesenchymal stem cells express CD56.

20. The method of claim 17, wherein said mesenchymal stem cells express protein C.
